(19) United States
(12) Patent Application Publication
(10) Pub. No.: US 2025/0104865 A1
(43) Pub. Date: Mar. 27, 2025

(54) DIAGNOSTIC SYSTEM, MEDICAL COOPERATION SYSTEM, AND OPERATION METHOD OF DIAGNOSTIC SYSTEM

(71) Applicant: FUJIFILM Corporation, Tokyo (JP)

(72) Inventor: Takeshi Yatsuo, Tokyo (JP)

(21) Appl. No.: 18/830,024

(22) Filed: Sep. 10, 2024

(30) Foreign Application Priority Data
Sep. 22, 2023 (JP) 2023-157485

(51) Int. Cl. G16H 50/20; G16H 30/40; G16H 40/40

(52) U.S. Cl. CPC G16H 50/20; G16H 30/40; G16H 40/40

(57) ABSTRACT

An imaging available time inquiry unit inquires of a plurality of imaging hospitals in a storage unit about imaging available time slots on the day of the imaging request and receives imaging available times from the imaging hospitals. An imaging available time display unit displays, on a display unit, the imaging available time slots received from the plurality of imaging hospitals and a moving time to the imaging hospital. A selection/reservation unit selects one of the imaging available times displayed by the imaging available time display unit and transmits the selected imaging available time to the imaging hospital to make a reservation. A medical image reception unit receives a medical image obtained by capturing the patient who has moved from the diagnostic hospital, from the imaging hospital reserved by the selection/reservation unit.

### CROSS-REFERENCE TO RELATED APPLICATIONS

The present application claims priority under 35 U.S.C. § 119 to Japanese Patent Application No. 2023-157485, filed Sep. 22, 2023. Each of the above application(s) is hereby expressly incorporated by reference, in its entirety, into the present application.

### BACKGROUND OF THE INVENTION

### 1. Field of the Invention

The present invention relates to a system that enables a patient to receive a diagnosis based on image capturing and image interpretation in a short time.

### 2. Description of the Related Art

In order to facilitate cooperation in a case in which a patient is introduced from one medical facility to another medical facility, an information processing system that connects a system of a regional hospital and a system of a core hospital via a network is disclosed in, for example, JP2021-99808A. In JP2021-99808A, the system of the regional hospital can generate a letter of introduction and transmit it to the system of the core hospital via the network, and the system of the core hospital can transmit reservation available dates for the core hospital to the system of the regional hospital. A patient or a doctor can make a reservation by selecting the reservation available date for the core hospital on a display screen of the system of the regional hospital.

### SUMMARY OF THE INVENTION

The information processing system of JP2021-99808A has an advantage in that it is easy to make a reservation at the core hospital from the system of the regional hospital, but it is still the same as the information processing system in the related art in that three visits to the core hospital are required for a first medical examination, imaging, and a diagnosis.

Specifically, as shown in FIG. 6B, in a case in which a patient visits a hospital to receive a first medical examination in order to receive a medical examination with some health anxiety (step S701), the patient is not imaged by a magnetic resonance imaging (MRI) apparatus or an X-ray CT apparatus on the day of the first medical examination unless the patient has an extremely acute symptom, and generally only makes a reservation for imaging (step S702), performs accounting for the medical examination (step S703), and then goes home.

Next, the patient goes to the hospital at the reserved date and time and is imaged (step S704). However, no diagnosis is made on the spot based on a captured image, and the patient makes a reservation for the next visit date and performs accounting for the imaging, and then goes home again (step S705).

Next, the patient goes to the hospital at the reserved date and time, and can know a diagnosis result based on an image interpretation result of the captured image (step S706), and performs accounting for the diagnosis (step S707).

Therefore, a three-day visit to the hospital for the first medical examination, the imaging, and the diagnosis is typical. In general, there is a reservation period for the three steps of the first medical examination, the imaging, and the diagnosis, so that a waiting period occurs, and a period required until all the steps are completed and the diagnosis is made is about several weeks.

In this way, the patient has to visit the hospital three times over three days and perform the accounting processing each time before the diagnosis is made and a treatment is started, which places a huge time burden on the patient. Further, it typically takes several weeks to get the diagnosis result, which places a great psychological burden on the patient as the patient has to spend several weeks in a state of anxiety.

An object of the present invention is to provide a system that makes it possible to perform a first medical examination, imaging, and a diagnosis on the same day on which a patient visits a hospital.

In order to achieve the above object, according to the present invention, there is provided a diagnostic system that issues a request for imaging via a communication line from a diagnostic hospital in which a diagnostician who diagnoses a patient is present to an imaging hospital in which a radiologist who captures a medical image of the patient is present. The diagnostic system comprises a storage unit, an imaging available time inquiry unit, an imaging available time display unit, a selection/reservation unit, and a medical image reception unit. The storage unit stores in advance a plurality of the imaging hospitals and a moving time required for the patient to move from the diagnostic hospital to the imaging hospital. The imaging available time inquiry unit inquires of the plurality of imaging hospitals in the storage unit about imaging available time slots on the day of the imaging request and receives imaging available times from the imaging hospitals. The imaging available time display unit displays, on a display unit, the imaging available time slots received from the plurality of imaging hospitals and the moving time to the imaging hospital, which is read out from the storage unit. The selection/reservation unit selects one of the imaging available times displayed by the imaging available time display unit and transmits the selected imaging available time to the imaging hospital to make a reservation. The medical image reception unit receives a medical image obtained by capturing the patient who has moved from the diagnostic hospital, from the imaging hospital reserved by the selection/reservation unit.

According to the present invention, it is possible to provide a diagnostic system that makes it possible to perform a first medical examination, imaging, and a diagnosis on the same day on which a patient visits a hospital.

### BRIEF DESCRIPTION OF THE DRAWINGS

FIG. 1 is a block diagram of a medical cooperation system according to an embodiment of the present invention.

FIG. 2 is a block diagram of the medical cooperation system according to the embodiment.

FIG. 3 is a flowchart showing an operation of the medical cooperation system according to the embodiment.

FIG. 4 is a screen example displayed on a diagnostic system of the medical cooperation system according to the embodiment.

FIG. 5 is a screen example displayed on the diagnostic system of the medical cooperation system according to the embodiment.

FIG. 6A is a flowchart showing movement of a patient in the medical cooperation system according to the embodiment, and FIG. 6B is a flowchart showing movement of a patient in the related art.

FIG. 7 is a block diagram showing a medical cooperation system of a comparative example.

### DESCRIPTION OF THE PREFERRED EMBODIMENTS

A medical cooperation system according to an embodiment of the present invention will be described with reference to the drawings.

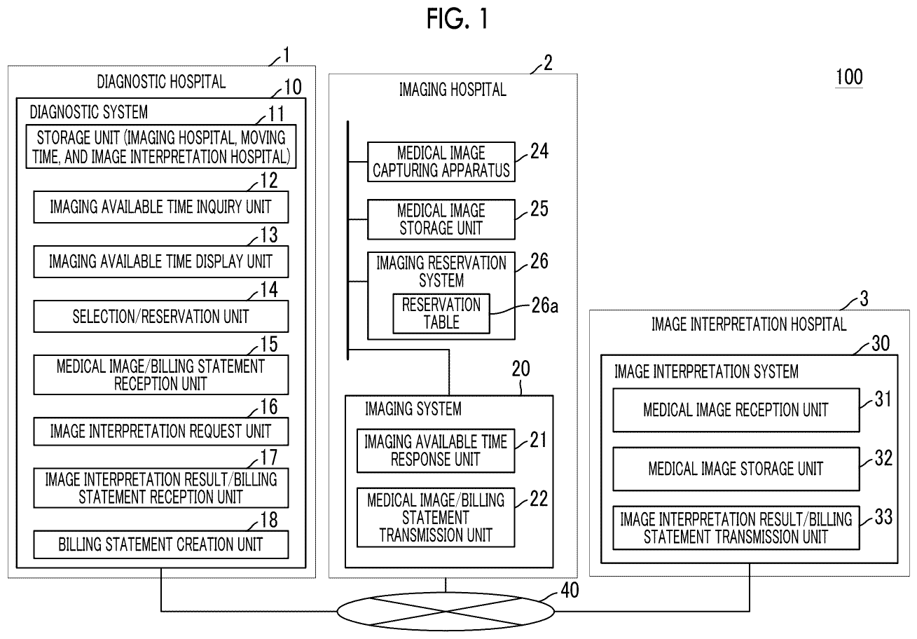

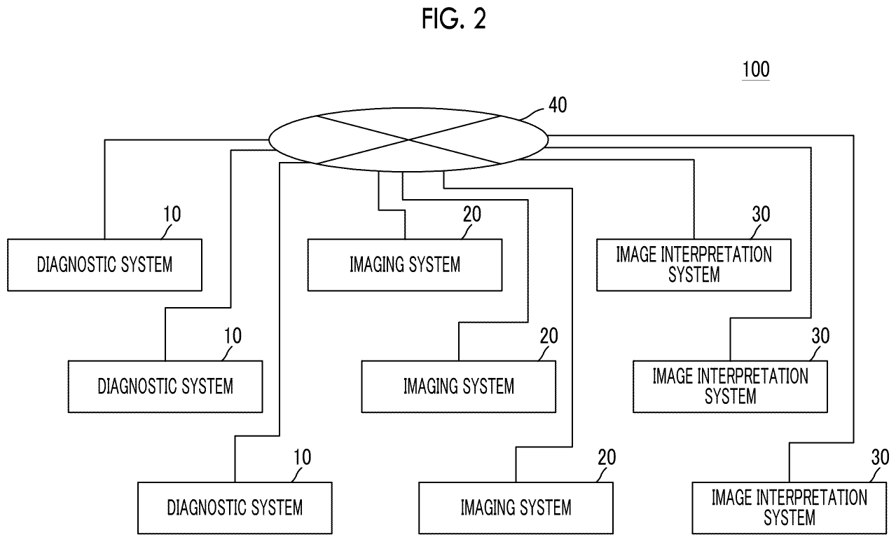

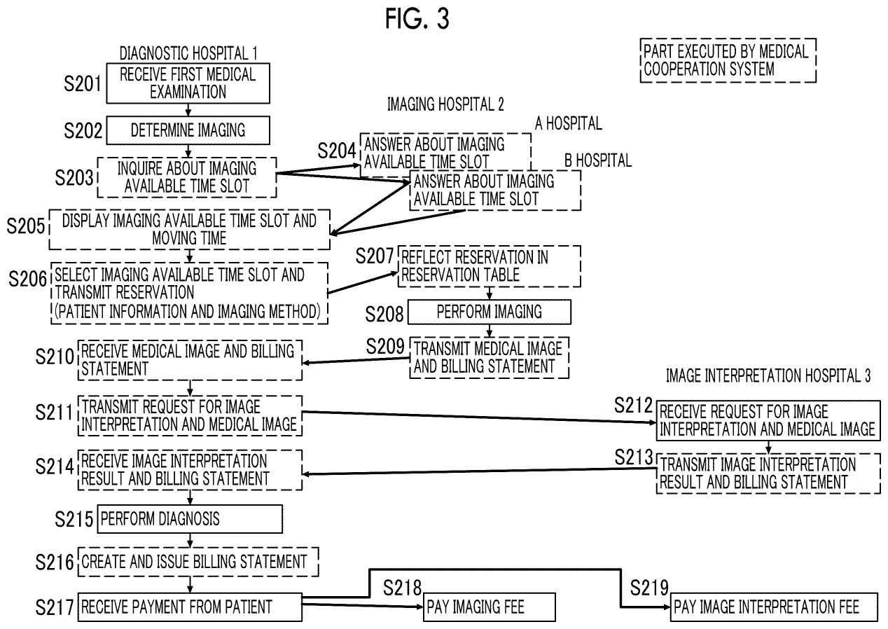

FIG. 1 is a block diagram showing a structure of a medical cooperation system 100 according to the present embodiment. FIG. 2 is an explanatory diagram showing a plurality of hospitals linked by the medical cooperation system 100. FIG. 3 is a flowchart showing an operation of each unit of the medical cooperation system 100.

As shown in FIG. 1, the medical cooperation system of the present embodiment includes a diagnostic system 10, an imaging system 20, and an image interpretation system 30.

The diagnostic system 10 is a system that is disposed in a hospital in which a diagnostician who diagnoses a patient is present (hereinafter, referred to as a diagnostic hospital 1). The diagnostic system 10 issues a request for imaging via a communication line 40 to a hospital in which a radiologist who is capable of capturing a medical image of the patient is present (hereinafter, referred to as an imaging hospital 2).

The imaging system 20 is a system that is disposed in the imaging hospital 2. The imaging system 20 receives the request from the diagnostic hospital 1, captures a medical image by using a medical image capturing apparatus 24 such as an MRI apparatus and an X-ray CT apparatus in the imaging hospital 2, and transmits the captured medical image to the diagnostic hospital 1 via the communication line 40.

The image interpretation system 30 is a system that is disposed in a hospital in which an image interpretation doctor is present (hereinafter, referred to as an image interpretation hospital 3). The image interpretation system 30 receives a request from the diagnostic hospital 1, interprets the medical image captured by the imaging hospital 2, and transmits an image interpretation result to the diagnostic hospital 1 via the communication line 40.

The diagnostic system 10 can inquire of the imaging systems 20 of a plurality of the imaging hospitals 2 about imaging available time slots on the day to make a reservation for capturing a medical image on the day. In this case, the diagnostic system 10 shortens a time until the capturing of the medical image is completed by taking into consideration a moving time from the diagnostic hospital 1 in which the patient is currently present to the imaging hospital 2, and making a reservation for a reservation time at which the imaging can be performed earliest or a reservation time with the shortest moving time.

The captured medical image may be received by the diagnostic system 10 and may be interpreted by the doctor in the diagnostic hospital 1, or the diagnostic system 10 may issue a request for image interpretation to the image interpretation system 30 of the image interpretation hospital 3. The doctor of the diagnostic hospital 1 diagnoses the patient by using an image interpretation result.

As a result, it is possible to perform three steps of a first medical examination, imaging, and a diagnosis on the same day on the day on which the patient visits the diagnostic hospital 1 for a medical examination, thereby reducing a burden on the patient and realizing early start of treatment. As shown in FIG. 1, the diagnostic system 10 of the diagnostic hospital 1 comprises a storage unit 11, an imaging available time inquiry unit 12, an imaging available time display unit 13, a selection/reservation unit 14, a medical image/billing statement reception unit 15, an image interpretation request unit 16, an image interpretation result/billing statement reception unit 17, and a billing statement creation unit 18.

The diagnostic system 10 is configured by a computer or the like comprising a processor such as a central processing unit (CPU) or a graphics processing unit (GPU), and a memory, and the processor reads and executes a program stored in the memory to realize functions of the imaging available time inquiry unit 12, the imaging available time display unit 13, the selection/reservation unit 14, the medical image/billing statement reception unit 15, the image interpretation request unit 16, the image interpretation result/billing statement reception unit 17, and the billing statement creation unit 18. In addition, the diagnostic system 10 can also be configured by hardware. For example, a circuit need only be designed to realize the function of each unit by using a custom IC such as an application specific integrated circuit (ASIC) or a programmable IC such as a field-programmable gate array (FPGA).

The storage unit 11 stores in advance information such as names and addresses of the plurality of imaging hospitals 2, a moving time required for the patient to move from the diagnostic hospital 1 to the imaging hospital 2, and information such as names and addresses of one or more image interpretation hospitals 3. These pieces of information may be information for introducing the imaging hospital 2 and the image interpretation hospital 3, which is transmitted to the diagnostic hospital 1 via a communication line or by mail, and which is registered in the storage unit 11 by an operator of the diagnostic hospital 1. In addition, the imaging hospital 2 and the image interpretation hospital 3 may register information for introducing their hospitals themselves on a database (not shown) connected to the communication line 40, and the operator of the diagnostic hospital 1 may download the information from the database and store the information in the storage unit 11.

The imaging available time inquiry unit 12 inquires of the imaging systems 20 of the plurality of imaging hospitals 2 registered in the storage unit 11 about imaging available time slots on the day of the imaging request and after the day of the imaging request, and receives imaging available times from the imaging systems 20 of the plurality of imaging hospitals 2.

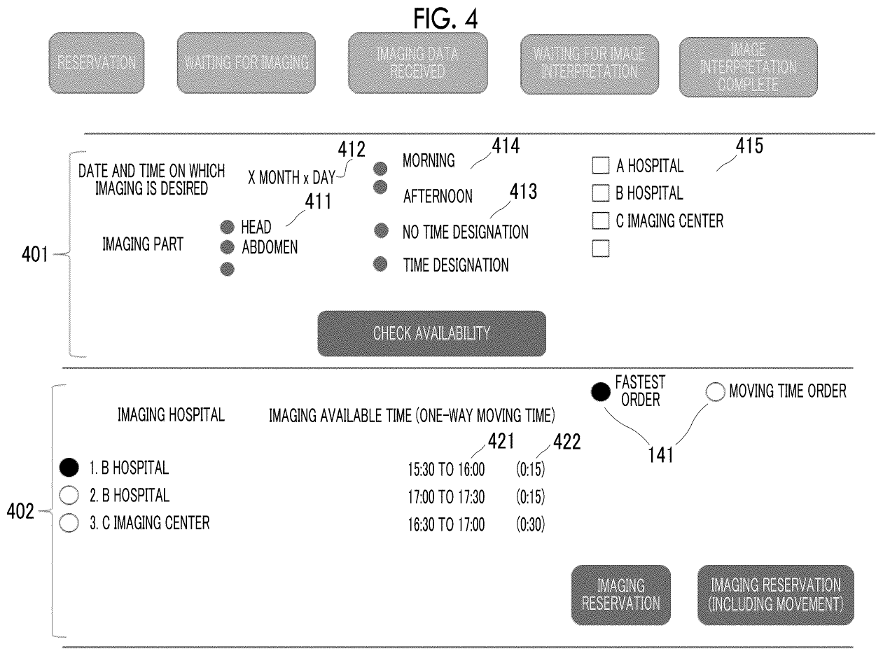

The imaging available time display unit 13 displays the imaging available time slots received from the plurality of imaging hospitals 2 and the moving time to the imaging hospital, which is read out from the storage unit, on a display unit connected thereto, for example, as shown in a screen example of FIG. 4.

The selection/reservation unit 14 selects one of the imaging available times displayed by the imaging available time display unit 13 and transmits the selected imaging available time to the imaging system 20 of the imaging hospital 2 to make a reservation. The selection/reservation unit 14 selects and reserves an imaging available time with the earliest imaging available time slot or with the shortest moving time from among the imaging available times. In this case, the selection/reservation unit 14 may comprise a reception unit 141 that receives from the operator which of the earlier imaging available time slot or the shorter moving time is to be prioritized in a case of making a selection, as shown in FIG. 4.

The medical image/billing statement reception unit 15 receives a medical image obtained by capturing the patient who has moved from the diagnostic hospital 1, and a billing statement for an imaging fee, from the imaging hospital 2 reserved by the selection/reservation unit 14

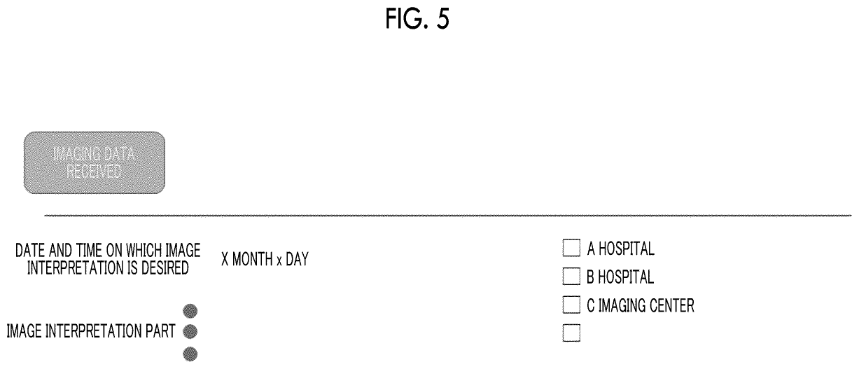

The image interpretation request unit 16 receives designation of an image interpretation part from the diagnostician, and transmits the medical image received by the medical image/billing statement reception unit 15 and the image interpretation part to the image interpretation system 30 of the image interpretation hospital 3 to issue a request for image interpretation, based on a screen example of FIG. 5.

The image interpretation result/billing statement reception unit 17 receives an image interpretation result and a billing statement for an image interpretation fee from the image interpretation system 30 of the image interpretation hospital 3.

The billing statement creation unit 18 issues a billing statement for a medical examination cost to the patient. In this case, the billing statement creation unit 18 creates a billing statement in which the imaging fee in the imaging fee billing statement received from the imaging hospital 2 and the image interpretation fee in the image interpretation fee billing statement received from the image interpretation hospital 3 are added to the medical examination cost, and bills the patient for the imaging fee of the imaging hospital 2 instead of the imaging hospital 2.

Meanwhile, as shown in FIG. 1, the imaging system 20 comprises an imaging available time response unit 21 and a medical image/billing statement transmission unit 22. As with the diagnostic system 10, functions of these units may be realized by software or by hardware.

The imaging hospital 2 is equipped with a medical image capturing apparatus 24, a medical image storage unit 25 that stores a medical image captured by the medical image capturing apparatus 24, and an imaging reservation system 26 that receives an imaging reservation in the hospital. These are connected to each other to construct an existing in-hospital system. A reservation table 26a showing an imaging available time is stored in the imaging reservation system 26.

In a case in which an inquiry about the imaging available time is received from the diagnostic system 10, the imaging available time response unit 21 refers to the reservation table 26a in the imaging reservation system 26, reads out the imaging available time within a range of the introduced date and time, and transmits the read-out imaging available time to the diagnostic system 10.

The medical image/billing statement transmission unit 22 reads out the medical image, which is captured by the medical image capturing apparatus 24 and stored in the medical image storage unit 25, from the medical image storage unit 25, and transmits the read-out medical image to the diagnostic system 10 together with the billing statement for the imaging fee.

As shown in FIG. 1, the image interpretation system 30 comprises a medical image reception unit 31, a medical image storage unit 32, and an image interpretation result/billing statement transmission unit 33. As with the diagnostic system 10, functions of these units may be realized by software or by hardware.

The medical image reception unit 31 receives the request for image interpretation and the medical image from the diagnostic system 10. The medical image is stored in the medical image storage unit 32. The doctor interprets the medical image stored in the medical image storage unit 32 to create an image interpretation result.

The image interpretation result/billing statement transmission unit 33 transmits the image interpretation result created by the doctor and the billing statement for the image interpretation fee to the diagnostic system 10.

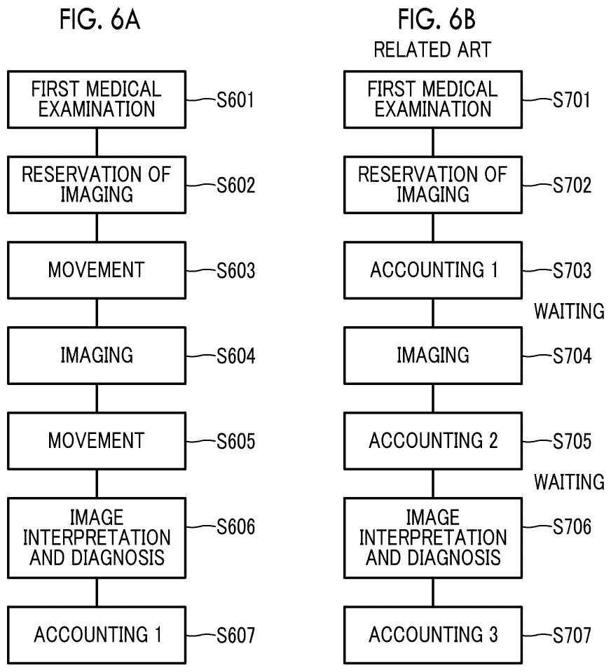

The movement of the patient in the medical cooperation system 100 according to the present embodiment will be described with reference to a flow of FIG. 6A.

In a case in which the patient receives a first medical examination at the diagnostic hospital 1 that the patient has visited for a medical examination (step S601) and the doctor determines that imaging is necessary, the diagnostic system 10 searches for a nearby imaging hospital 2 that can perform imaging on the same day and makes a reservation while taking into consideration a moving time (step S602).

The patient immediately moves on the same day and arrives at the imaging hospital 2 by the reserved time (step S603).

The imaging hospital 2 performs the imaging using the medical image capturing apparatus 24 (step S604).

The patient returns to the diagnostic hospital 1 (step S605).

The imaging system of the imaging hospital 2 transmits the captured medical image data and the billing statement for the imaging fee to the diagnostic system 10 of the diagnostic hospital 1. The diagnostic system 10 transmits the medical image data to the image interpretation system 30 in the image interpretation hospital 3. As a result, the image interpretation is executed while the patient is moving from the imaging hospital 2 to the diagnostic hospital 1. The image interpretation result is transmitted from the image interpretation system 30 to the diagnostic system 10. The doctor of the diagnostic hospital 1 receives the image interpretation result and the medical image from the diagnostic system 10, and performs a diagnosis. Upon returning to the diagnostic hospital 1, the patient is informed of the diagnosis by the doctor, and treatment is started (step S606).

The diagnostic system 10 of the diagnostic hospital 1 creates a billing statement including a diagnosis fee at its own hospital, the imaging fee at the imaging hospital 2, and the image interpretation fee at the image interpretation hospital 3, and bills the patient. As a result, the time and effort for creating the billing statement and making a payment are required only once (step S607).

By diagnosing and imaging the patient with such a flow, it is possible to reduce the time burden and psychological burden compared to the method in the related art. In addition, it is also possible to prevent the disease from progressing during the diagnosis or imaging for several weeks.

In addition, the diagnostic hospital 1 can give a diagnosis to the patient on the same day even though it does not own an expensive medical image capturing apparatus 24 such as an MRI or an X-ray CT apparatus, so that it is possible to provide prompt medical care while reducing the cost burden on the diagnostic hospital 1.

In addition, by utilizing the imaging hospital 2 outside and the image interpretation doctor of the image interpretation hospital 3, the diagnostician of the diagnostic hospital 1 can make a diagnosis even in cases that he or she has not experienced before, without having to simply refer the case to a higher-level hospital.

The imaging hospital 2 can effectively use an operating rate of the equipment owned and skills of the radiologist or technician without leaving them idle. The image interpretation doctor of the image interpretation hospital 3 can perform the image interpretation regardless of whether a region where the image interpretation hospital 3 is located is close to or far from the diagnostic hospital 1, and can effectively use image interpretation skills without leaving them idle.

From the viewpoint of the entire region, it is possible to reduce wasteful spending on medical care and to provide medical care with higher quality.

The operation of the medical cooperation system 100 will be described with reference to FIG. 3.

### Steps S201 and S202

The patient visits the diagnostic hospital 1 and receives a first medical examination by the diagnostician (step S201). In a case in which the diagnostician determines that it is necessary to capture a medical image (step S202), the diagnostician inputs, on an inquiry screen of FIG. 4 displayed by the imaging available time inquiry unit 12 of the diagnostic system 10, an imaging part, a date of the day on which the imaging is desired, presence or absence of a time slot, a time slot, and designation of the imaging hospital 2 to be inquired of, respectively, in regions 411 to 415 of the display screen.

### Step S203

The imaging available time inquiry unit 12 of the diagnostic system 10 transmits the imaging part, the date of the day, the presence or absence of the desired time slot for the imaging, and the time slot, which are input in the regions 411 to 414, to the imaging hospital 2 designated in the input region 415, and inquires of the imaging hospital 2 about the imaging available time slot.

### Step S204

The imaging available time response unit 21 of the imaging system 20 of each of the plurality of imaging hospitals 2 receives the inquiry about the imaging available time slot transmitted in step S203. The imaging available time response unit 21 refers to the reservation table 26a in the imaging reservation system 26, reads out the imaging available time slot within a range of the introduced date and time, and transmits the read-out imaging available time to the diagnostic system 10.

### Step S205

The imaging available time inquiry unit 12 of the diagnostic system 10 receives the imaging available time slot from the imaging available time response unit 21 of the imaging system 20.

The imaging available time display unit 13 of the diagnostic system 10 displays the imaging available times received from the plurality of imaging hospitals 2 and the moving time to the imaging hospital 2 read out from the storage unit 11, for example, in regions 421 and 422 of the screen example of FIG. 4. In this case, the imaging available time display unit 13 may display the imaging available times by excluding any time when the current time point plus the moving time has passed the start time.

### Step S206

The selection/reservation unit 14 selects one of the imaging available times displayed by the imaging available time display unit 13 and transmits the request for imaging to the imaging system 20 of the selected imaging hospital 2 to make a reservation. In this case, the selection/reservation unit 14 receives from the operator, via the reception unit 141, which of the earlier imaging available time slot or the shorter moving time is to be prioritized for selection. In a case in which the selection of the earlier imaging available time slot is received via the reception unit 141, the selection/reservation unit 14 selects and reserves the imaging available time with the earliest imaging available time slot from among the imaging available times. In addition, in a case in which the selection of the shorter moving time is received via the reception unit 141, the selection/reservation unit 14 selects and reserves the imaging available time with the shortest moving time.

In this case, the selection/reservation unit 14 receives patient information, an imaging part, and an imaging method from the diagnostician, and simultaneously transmits the patient information, the imaging part, and the imaging method to the imaging system 20.

In this case, the selection/reservation unit 14 may make arrangements for transportation simultaneously with the transmission. For example, a taxi or a pick-up car of the imaging hospital 2 that has been reserved for the patient is requested to pick up the patient at the diagnostic hospital.

### Step S207

The imaging available time response unit 21 of the imaging system 20 of the imaging hospital 2 that has been requested to perform the imaging transcribes the imaging available time slot transmitted from the selection/reservation unit 14 in step S206 into the reservation table 26a of the imaging reservation system 26 in the hospital, thereby reflecting the reservation in the imaging reservation system 26 in the hospital.

### Step S208

As soon as the patient arrives, the doctor or the technician of the imaging hospital 2 captures a medical image using the medical image capturing apparatus 24. The captured medical image data is stored in the medical image storage unit 25.

### Step S209

The medical image/billing statement transmission unit 22 reads out the medical image, which is captured by the medical image capturing apparatus 24 and stored in the medical image storage unit 25, from the medical image storage unit 25, and transmits the read-out medical image to the diagnostic system 10 together with the billing statement for the imaging fee.

### Step S210

The medical image/billing statement reception unit 15 receives the medical image data transmitted in step S209 and the billing statement for the imaging fee.

### Step S211

The diagnostician of the diagnostic hospital 1 may interpret the medical image in the diagnostic hospital 1 or may issue a request for image interpretation to the image interpretation hospital 3. In that case, the diagnostician selects an image interpretation part on a screen as shown in FIG. 5. The image interpretation request unit 16 of the diagnostic system 10 transmits the medical image received by the medical image/billing statement reception unit 15 and the image interpretation part to the image interpretation system 30 of the image interpretation hospital 3 to issue a request for image interpretation. It is desirable that the image interpretation hospital 3 is a hospital that gives priority to the image interpretation for the diagnostic hospital 1.

### Step S212

The medical image reception unit 31 of the image interpretation hospital 3 receives the request for image interpretation and the medical image from the diagnostic system 10. The medical image is stored in the medical image storage unit 32.

### Step S213

The doctor of the image interpretation hospital 3 interprets the medical image stored in the medical image storage unit 32 to create an image interpretation result.

The image interpretation result/billing statement transmission unit 33 transmits the image interpretation result created by the doctor and the billing statement for the image interpretation fee to the diagnostic system 10.

### Step S214

The image interpretation result/billing statement reception unit 17 receives the image interpretation result and the billing statement for the image interpretation fee transmitted in step S213.

### Step S215

The doctor of the diagnostic hospital 1 performs a diagnosis based on the medical image received in step S210 and the image interpretation result received in step S214, and explains a diagnosis result to the patient who has returned from the imaging hospital 2.

### Step S216

The billing statement creation unit 18 creates and issues a billing statement in which the imaging fee in the imaging fee billing statement received from the imaging hospital 2 in step S210 and the image interpretation fee in the image interpretation fee billing statement received from the image interpretation hospital 3 in step S214 are added to the medical examination cost.

### Steps S217 to S219

The diagnostic hospital 1 receives payment from the patient and pays the imaging fee to the imaging hospital 2 and the image interpretation fee to the image interpretation hospital.

As shown in FIG. 2, in the medical cooperation system 100 according to the present embodiment, the plurality of diagnostic hospitals 1, the plurality of imaging hospitals 2, and the plurality of image interpretation hospitals 3 cooperate with each other, and thus the plurality of imaging hospitals 2 can receive the imaging request from the plurality of diagnostic hospitals 1. Therefore, the operating efficiency of the medical image capturing apparatus 24 can be increased.

In addition, in the image interpretation hospital 3, transmission and reception of the medical image are performed, so that it is not necessary for the patient to move, and only the image interpretation doctor is required. Therefore, it is also possible for the diagnostic hospital 1 to cooperate with an image interpretation hospital 3 located far away from the diagnostic hospital 1 or an image interpretation hospital 3 located overseas. In order to shorten the image interpretation time, it is desirable for the diagnostic hospital 1 to secure the image interpretation hospital 3 that gives top priority to interpreting the medical image of the hospital itself. Similarly to the imaging hospital 2, the diagnostic hospital 1 can predict an imaging end time of the imaging hospital 2 from the reserved imaging time, inquire of the image interpretation hospital 3 about an image interpretation available time, and reserve an image interpretation time.

In addition, as shown in FIG. 2, in the medical cooperation system 100 according to the present embodiment, the diagnostic hospital 1, the imaging hospital 2, and the image interpretation hospital 3 are connected in a peer-to-peer type topology, and a server that controls the entire system is not required. Therefore, as long as the communication line is connected, it is possible for a hospital in a country on a back side of the earth to participate as the image interpretation hospital 3.

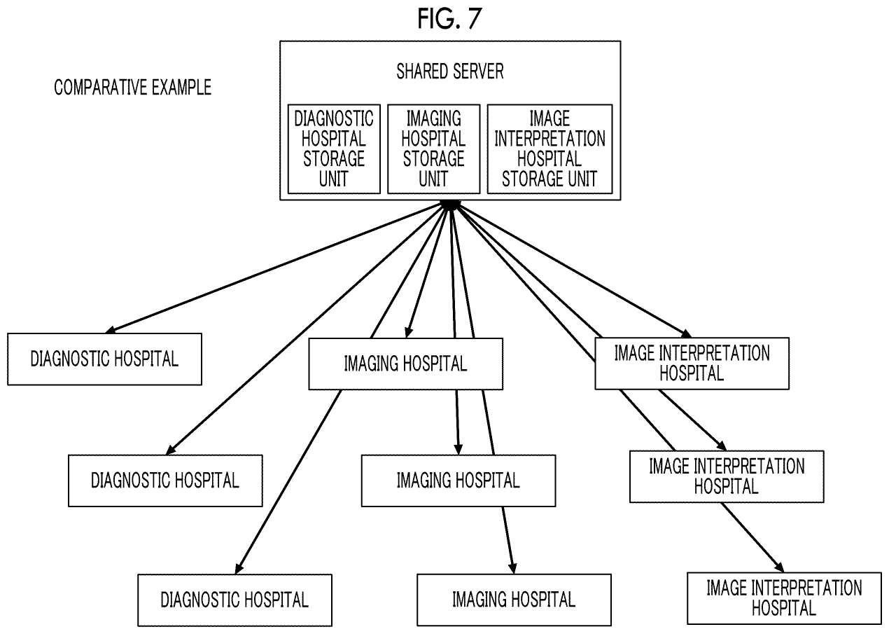

As a comparative example, in a case in which a system similar to the present embodiment is configured in a star type topology system in which a plurality of hospital systems are connected to a shared server, a configuration shown in FIG. 7 is obtained. In the system of FIG. 7, the shared server centrally manages the diagnostic hospital, the imaging hospital, and the image interpretation hospital. Such a system has advantages in that processing is easy, and it is easy to extract a global optimal solution since the shared server performs optimization while grasping requests of all nodes under its jurisdiction. In addition, once an interface for each of the diagnostic hospital, the imaging hospital, and the image interpretation hospital sides is decided, it is possible to improve functionality only by updating the server side.

On the other hand, the system of FIG. 7 has the following disadvantages. First, it is not easy to keep up with a change. The number of the diagnostic hospitals, imaging hospitals, and image interpretation hospitals is not constant and is increased or decreased at any time. The configuration of the shared use server has to be changed each time the number of the hospitals is increased or decreased.

Secondly, there is a problem of bearing a cost of maintaining the system itself. Assuming that initial members bear the costs of the initial construction fairly, it is quite difficult to determine what kind of costs should be borne by the participating members who have joined or left later. For example, it is difficult to jointly maintain this server unless there is a mechanism in which local governments are responsible for the construction and maintenance of the shared server.

Third, it is difficult to cope with a complex network. For example, in order for the image interpretation hospital located overseas to participate, it is necessary to construct the mechanism of FIG. 7 on a global network, but this is not an easy task. On the contrary, in a case in which the mechanism is configured on a global network, network outages may occur near a boundary of the network.

With respect to this, in the medical cooperation system 100 according to the present embodiment, the hospital that wants to participate can participate or can stop the participation with its own doctor by introducing any of the diagnostic system 10, the imaging system 20, or the image interpretation system 30. In addition, since the shared server is not required, the hospitals need only bear the costs of maintaining their own hospital's system. In addition, the type of the network does not matter as long as the communication line is connected.

What is claimed is:

1. A diagnostic system that issues a request for imaging via a communication line from a diagnostic hospital in which a diagnostician who diagnoses a patient is present to an imaging hospital in which a radiologist who captures a medical image of the patient is present, the diagnostic system comprising:
a storage unit in which a plurality of the imaging hospitals and a moving time required for the patient to move from the diagnostic hospital to the imaging hospital are stored in advance;
an imaging available time inquiry unit that inquires of the plurality of imaging hospitals in the storage unit about imaging available time slots on the day of the imaging request and that receives imaging available times from the imaging hospitals;
an imaging available time display unit that displays, on a display unit, the imaging available time slots received from the plurality of imaging hospitals and the moving time to the imaging hospital, which is read out from the storage unit;
a selection/reservation unit that selects one of the imaging available times displayed by the imaging available time display unit and that transmits the selected imaging available time to the imaging hospital to make a reservation; and
a medical image reception unit that receives a medical image obtained by capturing the patient who has moved from the diagnostic hospital, from the imaging hospital reserved by the selection/reservation unit.

2. The diagnostic system according to claim 1,
wherein the selection/reservation unit selects and reserves an imaging available time with an earliest imaging available time slot or with a shortest moving time from among the imaging available times.

3. The diagnostic system according to claim 1,
wherein the selection/reservation unit includes a reception unit that receives selection of the imaging available time by an operator, and selects and reserves the imaging available time received by the reception unit.

4. The diagnostic system according to claim 1, further comprising:
a billing statement creation unit that issues a billing statement for a medical examination cost to the patient,
wherein the medical image reception unit receives an imaging fee billing statement for an imaging fee from the imaging hospital together with the medical image, and
the billing statement creation unit creates a billing statement in which the imaging fee of the imaging hospital is added to the medical examination cost, and bills the patient for the imaging fee of the imaging hospital instead of the imaging hospital.

5. The diagnostic system according to claim 1, further comprising:
an image interpretation request unit; and
an image interpretation result reception unit,
wherein one or more image interpretation hospitals in which an image interpretation doctor is present and that are connected to the diagnostic hospital via the communication line are further registered in the storage unit,
the image interpretation request unit transmits the medical image received by the medical image reception unit to at least one of the image interpretation hospitals to request image interpretation, and
the image interpretation result reception unit receives an image interpretation result from the image interpretation hospital.

6. A medical cooperation system comprising:
the diagnostic system according to claim 1, wherein the diagnostic system, in which a diagnostic hospital in which a diagnostician who diagnoses a patient is present, issues a request for imaging to an imaging hospital in which a radiologist who captures a medical image of the patient is present via a communication line; and
an imaging system in which the imaging hospital in which the radiologist is present receives the request from the diagnostic hospital, captures a medical image, and transmits the medical image to the diagnostic hospital via the communication line.

7. The medical cooperation system according to claim 6, further comprising:
an image interpretation system in which an image interpretation hospital in which an image interpretation doctor is present receives a request from the diagnostic hospital, interprets the medical image, and transmits an image interpretation result to the diagnostic hospital via the communication line.

8. An operation method of a diagnostic system that issues a request for imaging via a communication line from a diagnostic hospital in which a diagnostician who diagnoses a patient is present to an imaging hospital in which a radiologist who captures a medical image of the patient is present, the operation method comprising:
inquiring of a plurality of the imaging hospitals about imaging available time slots on the day of the imaging request and receiving imaging available times from the imaging hospitals;
displaying, on a display unit, the imaging available time slots received from the plurality of imaging hospitals and a moving time to the imaging hospital which is read out from the storage unit;
selecting one of the displayed imaging available times and transmitting the selected imaging available time to the imaging hospital to make a reservation; and
receiving a medical image obtained by capturing the patient who has moved from the diagnostic hospital, from the reserved imaging hospital.
